(19) United States
(12) Patent Application Publication
(10) Pub. No.: US 2020/0206165 A1
(43) Pub. Date: Jul. 2, 2020

(54) SOLUBILIZATION OF CHLORHEXIDINE BASE, ANTISEPTIC OR DISINFECTANT COMPOSITIONS

(71) Applicant: LIMITED LIABILITY COMPANY "SUN SYSTEMS" [RU/RU], Moscow (RU)

(72) Inventors: Gennady Grigoryevich KARDASH, Moscow (RU); Jean-Christian ARTEMANN, Murcourt (FR); Alexander Yuryevich RYTSAREV Yuryevich RYTSAREV, Moscow (RU); Elena Nikolaevna HAPKINA, Moscow (RU)

(73) Assignee: LIMITED LIABILITY COMPANY "SUN SYSTEMS" [RU/RU], Moscow (RU)

(21) Appl. No.: 16/816,337

(22) Filed: Mar. 12, 2020

Related U.S. Application Data
(63) Continuation-in-part of application No. PCT/RU2018/000146, filed on Mar. 7, 2018.

(30) Foreign Application Priority Data
Oct. 23, 2017 (RU) 2017133879

(51) Int. Cl. A61K 31/155; A61K 9/48; A61P 31/06; A61K 47/18

(52) U.S. Cl. CPC A61K 31/155; A61K 9/4833; A61L 2/0082; A61K 47/183; A61K 47/186; A61P 31/06

(57) ABSTRACT

The invention discloses a chlorhexidine base and method for producing a solubilized form containing a chlorhexidine base in the presence of amine oxide. The invention is applicable in medicine and results in development of novel antiseptic or disinfectant compositions that have universal effect against all types of microorganisms, including those residing in biofilms, active against Mycobacterium tuberculosis, including strains of poly-resistant MDR, extremely resistant XDR and pan-resistant PDR, bacterial spores, fungi and viruses.

### CROSS-REFERENCE TO RELATED APPLICATIONS

The present application is a continuation-in-part of PCT/RU2018/000146 filed on Mar. 7, 2018. This application also claims the benefit of Russian Patent Application RU 2017133879 filed on Oct. 23, 2017. The contents of the abovementioned applications are incorporated by reference herein.

### FIELD OF TECHNOLOGY

The present invention relates to chlorhexidine base, creating a solubilized form containing chlorhexidine base and amine oxide. It belongs to medicine and pharmaceutical industry, in particular, to new antiseptic or disinfectant compositions that have universal effect against all types of microorganisms, including those residing in biofilms, active against Mycobacterium tuberculosis, including strains of poly-resistant MDR, extremely resistant XDR and pan-resistant PDR, bacterial spores, fungi and viruses.

### BACKGROUND

Chlorhexidine base belongs to the class of biguanides. Chemically, the chlorhexidine base is 1,1′-hexamethylene bis-[5-(4-chlorophenyl)-biguanide]. The formula of the chlorhexidine molecule is C₂₂H₃₀Cl₂N₁₀, and the structural formula is presented below.

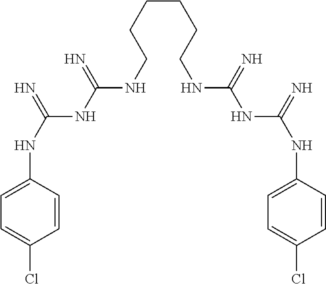 [SMILES: N=C(NCCCCCCNC(=N)NC(=N)Nc1ccc(Cl)cc1)NC(=N)Nc1ccc(Cl)cc1]

Chlorhexidine base is a white crystalline powder that is practically insoluble in water and alcohols. Given the poor solubility of chlorhexidine base, chlorhexidine is usually used as salts, mainly biacetate, bigluconate, or dihydrochloride. Bigluconate is the most soluble in water and alcohols, and, therefore, this form is widely used in urology, for manufacture of mouthwashes, gels and varnishes, etc. Chlorhexidine acts at the level of the cell membrane, increasing its permeability.

The first stage of action of chlorhexidine is its rapid adsorption on the microbial wall, which is explained by the presence of two main and symmetrical groups of chlorophenylguanide attached to the lipophilic chain of hexamethylene, which form a bicationic molecule that interacts with the bacterial surface. Conditions for their binding are most favorable in a neutral or slightly alkaline pH; amount of chlorhexidine adsorbed depends on the concentration of the agent. The outer layer of the bacterial cell wall carries a negative charge, usually stabilizing in the presence of cations such as Mg2+ and Ca2+; this is the basis for action of most cationic antiseptics, including chlorhexidine, which have high affinity for the bacterial cell wall.

Salts ofchlorhexidine, in particular bigluconate, are antiseptic with bactericidal and fungicidal effect. Although chlorhexidine bigluconate is not considered a virucide, there is a small activity against lipid membranes of viruses such as HIV, herpes 1 and 2, and influenza A. It can inhibit spore growth and has a bacteriostatic effect against some bacteria, but does not act on acid-resistant bacteria.

In low concentrations chlorhexidine bigluconate is a bacteriostatic agent, but when it is removed from the environment, functions of bacterial cell are restored.

As antiseptics and disinfectants, chlorhexidine salts have found their application in a variety of areas.

Dozens of aseptic and disinfecting compositions with chlorhexidine salts have been registered and produced. As an active component of chlorhexidine, bigluconate is part of the preparation “Citeal” (chlorhexidine bigluconate along with hexamidine and chlorocresol) produced by the French company “PIERRE-FABRE MED” and in the composition of the preparation “Plivasept” produced by the Croatian company “PLIVA” in the form of emulsions containing chlorhexidine bigluconate. 7 patents are devoted to the preparation and use of chlorhexidine derivatives and compositions based on it (patent WO 95/12395-Calgon Vestal Lab (USA); patent EP 306455-Warner Lambert Co (USA); patent WO 93/9770; Pierre Fabre (France)—patent EP 200607; “P. F. Cosmetique” (France) and others). Company “Ivoclar AG” (Germany) proposed to use chlorhexidine with HF in toothpastes (patents DE 415397, EP 539811, U.S. Pat. No. 5,393,516).

Phenyl derivatives and cyanide derivatives of chlorhexidine were considered as potential highly active fungicides. Company “Bayer” (Germany) developed a de-worming agent based on a composition of phenyl-guanidine-imidosol and tetrahydropyrimidine derivatives (patent EP 279343). Company “BASF” (Germany)—patents 2812945, 3922232), “American Cyanamid Co” (USA)—patents EP 406699, EP534501, U.S. Pat. No. 5,449,809), “Coro Jokko K. K.” (Japan.)-jap patents. 61-25706, 61-027, company “Hakko Chemical” (India).—(U.S. Pat. No. 5,116,838) developed new biocides. However, their industrial perspectives are very low due to existing high environmental requirements and difficulties in recycling phenolic and cyanide-containing waste.

Biocidal activity of chlorhexidine can be enhanced by introducing iodine compounds into composition (patent EP 473320).

Promising group of biocidal products based on guanidine-polyhexamethyleneguanidine and its derivatives presented in the patent of Russian Federation—No. 2351365 “Antiseptic composition “CHLORDIX” which contains a combination of well known, accessible and inexpensive antiseptics—chlorhexidine bigluconate and dioxidine. Activity was noted when acting on acid-resistant forms of bacteria, such as Helicobacter pylor and Micobacterium tuberculosis.

However, the description does not specify which types of strains of Micobacterium tuberculosis with what degree of resistance participated in the study. It may be assumed that strains sensitive to antibacterial drugs were used.

Each preparation containing chlorhexidine salt is enhanced by one or another substance to improve consumer quality, for example, alkyldimethylbenzylammonium chloride (catamine AB) or didecyldimethylammonium chloride, a non-ionic surfactant, quaternary ammonium salts, water-soluble oxyethylated monoalkyl phenols based on propylene trimers, etc. and acts against certain types of bacteria, but is ineffective against resistant Mycobacterium tuberculosis, bacterial spores, fungi shows little virucidal ability. In addition to this, according to modem research, resistance of microorganisms that are part of biofilms and account for up to 99% of all microorganisms in the natural habitat, is 100-1000 times higher than that of freely existing, so-called “planktonic” forms, which significantly reduces the effectiveness of antiseptics and disinfectants currently used.

Microorganisms have already developed resistance to many antiseptic or disinfectant compositions based on chlorhexidine salts. At the same time, the mechanism of action ofchlorhexidine on microbial cell and potential ofchlorhexidine base allows us to find new ways to solve the problem of resistance by obtaining other forms of chlorhexidine base, which are highly soluble in water and alcohol.

One way is to obtain chlorhexidine base solubilizers.

### SUMMARY OF THE INVENTION

The purpose of the invention is to create a well-soluble solubilizate of chlorhexidine base and aminoxide, to create antiseptic and/or disinfecting compositions based on this combination that have a universal effect against all types of microorganisms, including those inside biofilms, active against Mycobacterium tuberculosis, including poly-resistant (MDR), extremely resistant (XDR) and pan-resistant (PDR) strains, bacterial spores, fungi and viruses that have high safety level and economic affordability.

The problem of microorganism resistance is solved by a method of solubilization of chlorhexidine base, comprising encapsulating of chlorhexidine base in a presence of an amine oxide.

In another embodiment, the method is characterized by the fact that an amino acid is added during encapsulation to enhance solubilization process. In the preferred embodiment, the amine oxide is one from the following group: N-lauryl-N, N-dimethylaminoxide, N,N-dimethyldodecan-1-amine oxide, Lauryldimethylamine oxide, Tallowbis(2-hydroxyethyl)amine oxide, C12-18-alkyldimetylamine oxide, and the amino acid is one from the following group: leucine, tyrosine, serine, glutamine, asparagine, phenylalanine, alanine, lysine, arginine, histidine, glycine, cysteine, valine, proline, methionine, threonine, hydroxylysine.

In another aspect of the invention, the problem is solved by new antiseptic or disinfectant composition that comprises chlorhexidine base and an amine oxide, wherein this antiseptic or disinfectant composition inhibits growth of bacterial or fungal cells and is soluble in water or alcohol. Term “antiseptic or disinfectant” means that the composition can be used as an antiseptic composition or as a disinfectant composition. In another embodiment, the composition additionally comprises an amino acid. In yet another preferred embodiment, the amine oxide is one from the following group: N-lauryl-N, N-dimethylaminoxide, N,N-dimethyldodecan-I-amine oxide, Lauryldimethylamine oxide, Tallowbis(2-hydroxyethyl)amine oxide, C12-18-alkyldimetylamine oxide; and the amino acid is one from the following group: leucine, tyrosine, serine, glutamine, asparagine, phenylalanine, alanine, lysine, arginine, histidine, glycine, cysteine, valine, proline, methionine, threonine, hydroxylysine. In other embodiments, the amine oxide may be selected from the following group: Barlox-12i (“Lonza”), Minox L Lauramine oxide (<MIWON Commercial Co.>>), Aromox T/12, Tallowbis(2-hydroxyethyl) (<<Akzo Nobel>>), Empigen ODC 12-18-alkyldimetylamine Oxide (<<Hunstman corporation>>). Other amine oxides may be used in other embodiments.

In yet another embodiment, the composition is intended for therapeutic or prophylactic use against Micobacterium tuberculosis strains, including at least one of the following strain: poly-resistant MDR strains, extremely resistant XDR strains and pan-resistant PDR strains. In yet another embodiment, the composition disrupts biofilms formed from bacterial or fungal cells. In yet another embodiment, the composition is a concentrate and further comprises glycerin, a dye and water in the following ratio of components, mass fraction %: Chlorhexidine base-0.1-1.0%, Amine oxide-0.2-5.0%, Amino acid-0.05-2.0%, Glycerin-0.0-5.0%, Dye-0.01-0.1%, Water—up to 100%. In yet another embodiment, the composition is a concentrate and further comprises polyethylene glycol, a dye and water in the following ratio of components, mass fraction %: Chlorhexidine base-0.5-5.0%, Amine oxide-5.0-30.0%, Polyethylene glycol-0.2-3.0%, Amino acid-0.05-2.0%, Dye-0.01-0.1%, Water—up to 100%. In yet another embodiment, the composition is a concentrate and further comprises an alcohol, a dye and water in the following ratio of components, mass fraction %: Chlorhexidine base-0,5-65.0%, Amine oxide-0.2-5.0%, Amino acid-0.1-2.0%, Dye-0.01-0.1%, Alcohol-5.0-50.0%. In another aspect of the invention, the problem is solved by a method for inhibiting growth of bacterial or fungal cells, comprising contacting said cells with an antiseptic composition comprising chlorhexidine base and an amine oxide for a sufficient time, wherein the antiseptic composition is soluble in water or alcohol. In another preferred embodiment, the antiseptic composition additionally comprises an amino acid, quatemary ammonium compound (QAC), anti-corrosion additive and acidity regulator. The amine oxide may be selected from the following group: Barlox-12i (“Lonza”), Minox L Lauramine oxide (<MIWON Commercial Co.>), Aromox T/12, Tallowbis(2-hydroxyethyl) (<<Akzo Nobel>>), Empigen ODC12-18-alkyldimetylamine Oxide (<<Hunstman corporation>>).

The antiseptic composition of the invention has an antibacterial effect, including Mycobacterium tuberculosis, fungicidal, virucidal and sporicidal activity and is intended for aseptic hand treatment, pre-sterilization of medical devices (including flexible endoscopes and instruments), as well as for disinfection of patient care items, hygiene of medical staff and household purposes, does not cause corrosion of metal products.

### BRIEF DESCRIPTION OF THE DRAWINGS

FIG. 1 is the IR spectrum of solid samples of chlorhexidine base, chlorhexidine-amine oxide and glycine.

### DETAILED DESCRIPTION OF THE INVENTION

In the first aspect of the invention, it was found that chlorhexidine base and amine oxide associates form morphological structures—solubilizates that are well-soluble in water and alcohol. Solubilization is a particular mode of bringing into solution substances that are otherwise insoluble in a given medium, involving the previous presence of a colloidal solution (solubilizer) whose particles take up and incorporate within themselves the otherwise insoluble material.

It was found that the chlorhexidine base and amino oxide solubilizate constitute micellar or lamellar plate formations—liquid crystals.

The data of UV and NMR spectroscopy, EPR showed that when solubilizing in the plate micelles chlorhexidine base enters the micelles, located between the hydrocarbon ends and, thereby, spreads the layers of the molecular chains, forms dendritic or liquid crystals. A new morphological structure of dendritic crystals is formed, distinguishable from the morphology of the initial components of chlorhexidine base and amine oxide (glycine). The emergence of a new crystalline modification is preceded by an ordered state of molecules in a solution with electrostatic and hydrogen intermolecular bonds, which leads to the appearance of an abnormally high viscosity at a low concentration of components (0.1-1%).

New morphological structure unexpectedly showed synergistic biocidal activity against microorganisms, including Mycobacterium tuberculosis of the multiresistant MDR strain, the extremely resistant XDR and against the pan-resistant PDR strain, bacterial spores, fungi and virucidal activity.

To understand the synergistic mechanism, studies were carried out using various physicochemical methods that showed the protonation of the imino group of the chlorhexidine molecule and the presence of a new morphological structure of dendritic, liquid crystals in dilute solutions of chlorhexidine base, amine oxide (Barlox 12) and amino acide (glycine).

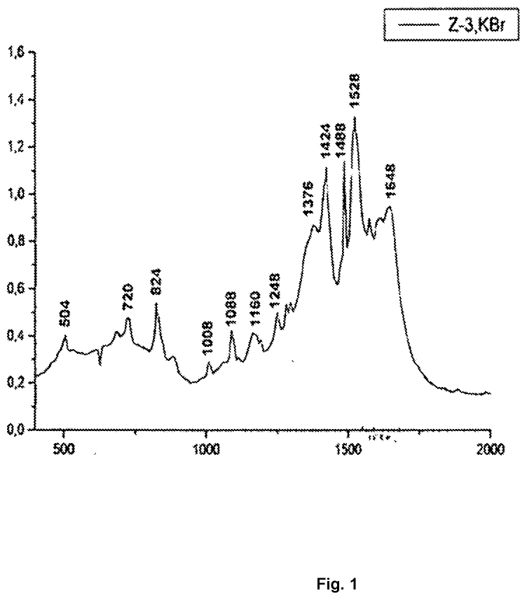

The IR spectrum of solid samples of chlorhexidine base, chlorhexidine-amine oxide and glycine solubilizate was obtained on an M-40 instrument (Germany) in the form of powders pressed with EHF (FIG. 1). Analyzing the IR spectrum (FIG. 1) it can be argued that carboxyl groups of glycine are in the charged form (COO⁻).

In the field of absorption of —COOH group at 1700 cm⁻¹ bands are not found in the spectrum of glycine, nor in the spectrum of the mixture of glycine with chlorhexidine base. At the same time, in the field of COO⁺ absorption groups at 1600 cm⁻¹ and 1400 cm⁻¹ there are strong bands in both spectra.

These data prove the mechanism of dissolution of chlorhexidine base solubilizate in aqueous solutions, which occurs by protonation of chlorhexidine base.

UV spectrum of chlorhexidine solubilizate obtained by UV spectrophotometer M-80 (Germany) confirmed the conclusion of IR spectroscopy data on the cause of dissolution of chlorhexidine base in amine oxide solution in the presence of an amino acid (glycine), which is due to the transfer of proton to chlorhexidine base.

Thus, microorganism resistance problem is solved by a method of solubilization of chlorhexidine base by encapsulation of chlorhexidine base in a presence of an amine oxide. Biocidal action of the composition can be improved by addition of amino acids, quaternary ammonium salts and other substances.

The new morphological structure showed synergistic biocidal activity towards all types of microorganisms, including those contained in biofilms and Mycobacterium tuberculosis of the poly-resistant MDR strain, extremely resistant XDR and pan-resistant PDR strain, bacterial spores, fungi and viruses.

In the second aspect of the invention, antiseptic and disinfecting compositions that have universal effect against all types of microorganisms, including those inside biofilms and active towards Mycobacterium tuberculosis, including strains of poly-resistant MDR, extremely resistant XDR and pan-resistant PDR, bacterial spores, fungi and viruses were obtained from chlorhexidine base solubilizate and auxiliary substances. The antiseptic composition comprises chlorhexidine base, an amine oxide and an amino acid, wherein the antiseptic composition inhibits growth of bacterial or fungal cells and is soluble in water or alcohol.

The following examples are provided for the purpose of disclosing the characteristics of this invention and should not be considered in any way as limiting the scope of the invention.

### Example 1. Preparation of Disinfectant Composition

To obtain 100 ml of an aseptic composition in the form of a concentrate at room temperature, 1 ml of amine oxide (Barlox-12), company “Lonza”, is dissolved in 100 ml of water, 0.15 g of chlorhexidine base, 0.05 g of glycine, 0.01 g of E133 dye. The resulting composition with a pH of 7.5-8 is an aseptic concentrate.

### Example 2. Preparation of Disinfectant Composition

It is carried out similarly to example 1, but the composition includes 0.5 g of chlorhexidine base, 3.5 ml of Barlox-12, 1.5 g of alanine, 2 ml of glycerol, and 0.05 g of carmoisine (E122).

### Example 3. Preparation of Disinfectant Composition

It is carried out similarly to example 1, but the composition includes 1 g of chlorhexidine base, 5 ml of Barlox-12, 2.0 g of glutamine, 2 ml of glycerol, 0.05 g of anthraquinone dye.

Series of experiments was performed which confirmed high activity of antiseptic compositions against Micobacterium tuberculosis, including the poly-resistant MDR strain, the extremely resistant XDR, which is not sensitive to classes 1, 2 and 3 of antibacterial drugs, and the pan-resistant PDR, which is not sensitive to all known antibacterial drugs of all classes. The laboratory strain of Micobacterium tuberculosis H37Rv and clinical (wild) strains of MDR, XDR, and PDR isolated from diagnostic material of patients with pulmonary tuberculosis who are being treated in the hospital of the Central Research Institute of Tuberculosis of the Russian Academy of Medical Sciences were used as biotests. Two clinical strains have been described as sensitive in relation to anti-TB drugs and are designated Sensitive −1 and Sensitive −2. The other three with varying degrees of resistance are labeled MDR, XDR, and PDR strains.

In accordance with the work plan, the culture of the selected strains of Mycobacterium tuberculosis was grown for 21 days on a dense Levenstein-Jensen nutrient medium (international standard).

Suspension of mycobacteria with an initial concentration of 5*10⁸, i.e. containing 500 million microbial bodies in 1 ml of solution, was prepared from the grown culture. The test substance was a ready-to-use solution. The time of exposure to Mycobacterium tuberculosis culture was 30 minutes and 60 minutes.

The experiment was performed using a modified immersion method. The ratio of suspension of Mycobacterium tuberculosis, and aseptic compositions of Example 2 (diluted in the ratio of 1 ml of the concentrate into 100 ml of water) was 1:1, i.e. in the test tube was poured 2 ml of a suspension of Mycobacterium tuberculosis of various strains and 2 ml of solution concentrate in an aseptic composition. Thus, the concentration of the bacterial suspension used in the work was 5*10⁴. This concentration of microbial bodies in the suspension corresponds to the required number of mycobacteria to detect them in the diagnostic material when painting smears using the ZIL-Nielsen method (light microscopy). After a given exposure, the mixture was centrifuged for 15 minutes at 3000 rpm. The resulting sediment was washed twice with distilled water. The seeding dose was 0.5 ml of sediment per tube with a dense Finn-2 nutrient medium and 0.5 ml of sediment per tube with a liquid Middlebrook 7N9 nutrient medium. Test tubes, in which suspension of mycobacteria used for test strains, was sown and was not treated with the antiseptic composition, were used for control. Crops on dense media were incubated in a thermostat at a temperature of 37° C.; crops on liquid media were incubated in the automated Bactec MGIT-960 system. Results on a dense nutrient medium were collected on the 28th, 42nd and 70th day from the moment of sowing. Results on the liquid nutrient medium were recorded on the Bactec MGIT-960.

Table 1 shows the results of a cultural study of the antibacterial composition. A microbiological study of this composition for bactericidal activity against Mycobacterium tuberculosis, including the poly-resistant MDR strain, the extremely resistant XDR, which is not sensitive to classes 1, 2 and 3 of antibacterial drugs, and the pan-resistant PDR, which is not sensitive to all known antibacterial drugs of all classes, was carried out in accordance with the regulatory and technical documentation and the order of the Ministry of health of the Russian Federation no. 109 of 21 Mar. 2003, Appendix No. 1, sanitary regulations SP 1.2.731-99.

TABLE 1
Results of a cultural study of the action of an antibacterial
composition containing chlorhexidine base.

| | antiseptic composition | | | | | |
|---|---|---|---|---|---|---|
| | containing chlorhexidine base | | | | | |
| | Dense medium | | | Liquid medium | | |
| Strains | 70 days | | | 42 days | | |
| Mycobactérium | 30 | 60 | | 30 | 60 | |
| tuberculosis | min | min | Control | min | min | Control |
| H37RV- | — | — | >100 | — | — | >100 |
| laboratory | | | | | | |
| sensitive-1 | — | — | >100 | — | — | >100 |
| clinical | | | | | | |
| sensitive-2 | — | — | >100 | — | — | >100 |
| clinical | | | | | | |
| MDR-strain- | — | — | >100 | — | — | >100 |
| clinical | | | | | | |
| XDR-strain- | — | — | >100 | — | — | >100 |
| clinical | | | | | | |
| PDR-strain- | — | — | >100 | — | — | >100 |
| clinical | | | | | | |

Where: (>100)—growth of Mycobacterium tuberculosis colonies on dense and liquid nutrient media; (−)—no growth of Mycobacterium tuberculosis on dense and liquid nutrient media; The results of these studies have shown high and stable activity against virulent clinical strains of Mycobacterium tuberculosis with varying degrees of resistance.

On day 70 or more from the time of seeding, no growth of colonies of the poly-resistant MDR strain, the extremely resistant XDR, and the pan-resistant PDR was detected.

### Example 5

Variants of concentrates of disinfecting compositions contain chlorhexidine base, amine oxide, quaternary ammonium compound (QAC), acidity regulator, anti-corrosion additive, thickener, auxiliary substances, with the following ratio of components, wt. %:

Variant 1

Chlorhexidine base-1.0-2.0%

Amine oxide-1.0-15.0%

Quaternary ammonium compound (QAC)—5.0-10.0%

Acidity regulator-0.1-1.0%

Anti-corrosion additive-0.1-1.0%

Dye—0.01-0.1%

Water—up to 100%

Preferably, alkyldimethylbenzylammonium chloride (Catamine A or B) is used as an QAC.

Preferably, triethanolamine, Trilon A, B, or C are used as anti-corrosion additive.

Preferably, the E133 dye is used as the dye.

Variant 2—concentrate of the disinfectant composition contains chlorhexidine base, amine oxide, amino acid, auxiliary substances, with the following ratio of components, wt. %:

Chlorhexidine base—0.5-5.0%

Amine oxide—5.0-30.0%

Polyethylene glycol—0.2-3.0%

Amino acid—0.1-2.0%

Dye—0.01-0.1%

Water—up to 100%

N-lauryl-N, N-dimethylaminoxide (Barlox-12), catalog of the company “Lonza” is used as amine oxide.

Leucine, tyrosine, terine, glutamine, asparagine, phenylalanine, alanine, lysine, argenine, histidine, glycine, cysteine, valine, proline, methionine, threonine, and hydroxylysine are used as amino acids. Triarylmethane dye (E133) is used as a dye.

Variant 3—concentrate of the disinfectant composition contains chlorhexidine base, aminoxide, amino acid, auxiliary substances with the following ratio of components, wt. %:

Chlorhexidine base-0.5-65.0%

Amine oxide-0.2-5.0%

Amino acid-0.1-2.0%

Dye—0.01-0.1%

Alcohol—5.0-50.0%

N-lauryl-N, N-dimethylaminooxide (“Barlox-12”, “Lonza” company catalog) is used as an amine oxide.

Leucine, tyrosine, terine, glutamine, asparagine, phenylalanine, alanine, lysine, argenine, histidine, glycine, cysteine, valine, proline, methionine, threonine, and hydroxylysine are used as an amino acid.

Triarylmethane dye (E133) is used as dye.

Ethanol, propanol, and methanol are used as alcohol.

Preparation of concentrates of disinfectant compositions of Variants 1, 2 and 3 is carried out similarly to Example 1, by mixing the components at room temperature in the specified proportions of active substances.

### Example 6

To study disinfectant activity against strains of Mycobacterium tuberculosis, against gram-negative and gram-positive bacteria, fungi concentrates of disinfectant compositions based on Variants 1, 2, and 3 were diluted in water in a ratio of 1:100.

Bacterial agents frequently occurring in surgical practice, such as Enterobacter spp., Citobacter spp. Klebsiella, Escherichia coli, Pseudomonas aeruginosa, Staphylococcus aureus, Poliomielitys and Candida were sown on nutrient media using conventional methods and evaluated in accordance with the requirements of the regulatory documentation “Methods of laboratory research and testing of disinfectants for efficiency and safety evaluation” (p 4.2.2643-10) and the regulatory documentation “Normative indicators of safety and effectiveness of disinfectants subject to control under mandatory certification”, No. 01-12/75-97

The study was conducted at the Federal research Institute for disinfection of Rospotrebnadzor of the Russian Federation. Data on the disinfecting activity are shown in Table 2.

TABLE 2

| | Efficiency of disinfection of test objects contaminated with test microorganisms | | | | | | |
|---|---|---|---|---|---|---|---|
| | (in % to 100% hygienic standards) | | | | | | |
| | | Candida | Staphylococcus | | | Bacillus | |
| Variants of | M. Terrae | Albicans | aureus | Microsporum | Escherichia | cereus | |
| compositions | strain DSM/ | (strain 15)/ | (strain 906)/ | gypseum/ | coli/ | (strain 96)/ | Poliomyelitis)/ |
| (1% solution) | exposure time | exposure time | exposure time | exposure time | exposure time | exposure time | exposure time |
| Antiseptic | 100%/ | 100%/ | 100%/ | 100%/ | 100%/ | 100%/ | 100%/ |
| composition | 30 min. | 30 sec | 30 sec | 1 min | 30 sec | 3 min | 30 min |
| 1 | 100%/ | 100%/ | 100%/ | 100%/ | 100%/ | 100%/ | 100%/ |
| | 60 min. | 30 sec | 30 sec | 1 min | 30 sec | 5 min | 60 min |
| 2 | 100%/ | 100%/ | 100%/ | 100%/ | 100%/ | 100%/ | 100%/ |
| | 30 min. | 45 sec | 30 sec | 1 min | 30 sec | 3 min | 30 min |
| 3 | 100%/ | 100%/ | 100%/ | 100%/ | 100%/ | 100%/ | 100%/ |
| | 60 min. | 45 sec | 30 sec | 1 min | 30 sec | 3 min | 60 min |

Thus, the claimed antiseptic and disinfection compositions have an universal effect against all types of microorganisms, including Mycobacterium tuberculosis of a poly-resistant strain of MDR, an extremely resistant strain of XDR and against a pan-resistant strain of PDR, as well as bacterial spores, fungi and viruses.

### Example 7. Testing Antiseptic, “Hexanidine Sept”, Obtained Via Solubilization (Encapsulation) of Chlorhexidine Base, Methodological Aspects

Antiseptic composition “Hexanidine Sept” is a water solution of the disinfectant concentrate “Hexanidine” containing 0.1% of chlorhexidine base, 0.12% of an amine oxide, 0.375% of Quaternary ammonium compound (QAC) and excipients.

The following research was conducted in the “State Scientific Center for Applied Microbiology” based in Obolensk. All works are performed in accordance with the requirements of sanitary and epidemiological rules set out in the following documents:
“Sanitary and epidemiological rules” SR 1.3.2322-08 “Safety of working with microorganisms of groups III-IV of pathogenicity (danger) and pathogens of parasitic diseases” [6].
Guidelines 4.2.1890-04 from 4 Mar. 2014 “Determining the sensitivity of microorganisms to antibacterial compositions.”

### Cultivation of Microorganisms.

For cultivation of microorganisms, agar and Muller-Hinton broth (Himedia, Mumbai, India), agar and GRM broth (FBUN SSC PMB, Obolensk, Russia) were used. Cultivation of planktonic forms of microorganisms was carried out for 20-24 hours at a temperature of 37° C. Cultivation of microbial biofilms on dense nutrient media was carried out for 168 hours at a temperature of 37° C.

### Evaluation of the Antibacterial Activity of “Hexanidine Sept”

Antibacterial activity of the composition “Hexanidine Sept” against test strains of pathogens of nosocomial infections was conducted as follows:

a) Method of serial dilutions in broth (Guidelines 4.2.1890-04):
test tubes containing 4 ml of nutrient broth and double dilutions of the composition “Hexanidine Sept” from 2.5 to 0.001% of the concentrate, sown 0.02 ml of night bacterial culture at a concentration of 10⁷ CFU/ml, incubated at 37° C. the presence of bacterial growth was taken into account visually by the presence of turbidity in the test tube. The minimum suppressive concentration (MSC) took the minimum concentration of the drug, in which there was no growth after 24 hours of incubation, the minimum bactericidal concentration (MBC)—72 hours

b) Method of serial agar dilution (by microdrops): on the surface of a nutrient agar containing serial dilutions of the composition “Hexanidine Sept” (from 1.25 to 0.002% of the concentrate), 10 μl of bacterial suspension in a concentration of 107 CFU/ml is applied and left until the drop is completely absorbed. The results obtained were taken into account by the presence of culture growth at the place of application of the microdrops after 24 h (MSC) and 72 h (MBC) incubation at a temperature of 37° C.

c) method of applicator: the surface of the nutrient agar that does not contain antibacterial additives, were seeded with 0.1 ml of a suspension of the studied culture at a concentration of 10′ CFU/ml. The cultures were cultivated at 37° C. for 24 hours, after which sterile cellulose applicator (7×7 mm) was applied on the surface of bacterial lawn for 2-3 min with sterile forceps. Then the applicator with the culture print was transferred with sterile tweezers to the surface of the agar in another Petri dish with successive dilutions of the composition “Hexanidine Sept” (from 1.25 to 0.002% of the concentrate), in the direction—“down the bacterial print”. The results were calculated taking into account test culture growth on and around the applicator after 72 hours of incubation at a temperature of 37° C. For MBC, the minimum concentration of the composition was taken, at which there was no growth of the culture. The MSC was not determined, since after 24 hours of cultivation, an “inertial” growth of the test culture was observed on the surface of the applicator.

Evaluation of Resistance Formation from Pathogens of Hospital-Acquired Infections to the Composition (“Hexanidine Sept”) Obtained by Solubilization (Encapsulation) of Chlorhexidine Base.

Selection of microorganisms resistant to compositions “Hexanidine Sept” was carried out by successive re-seeding bacterial cultures to a new portion of the nutrient broth containing stepwise increasing composition concentrations starting with the concentration of half of the MBC. To do this, we prepared a series of double dilutions of the composition in the volume of 4 ml of nutrient broth and injected 50 mics of bacterial culture into each tube and incubated at a temperature of 37° C. After 2-3 days of incubation, a portion of 50 mics was taken from a tube with the maximum concentration of the drug, in which bacterial growth was observed, and transferred to a new series of tubes with a higher concentration of the composition. The selection process continued until the increase in the MBC value for this culture was stopped for six repeats.

### Example 8. Testing Antiseptic “Hexanidine Sept” Obtained by Solubilization of Chlorhexidine Base, Results

Strains of Microorganisms that Cause ENT Infections of the Upper Respiratory Tract.

During the work, a collection of microorganisms that are pathogens of nosocomial infections was formed:
reference strains of bacteria K. pneumoniae ATCC 700603, P. aeruginosa ATCC 27853, A. baumannii ATCC 15308, E. cloacae ATCC BAA-2468, S. aureus No. 906, C. albicans ATCC 10231 and M. terrae TCM 1450 obtained from the State collection of pathogenic microorganisms “SCPM-Obolenslo>>;
clinical strains of K. pneumoniae, B-2523/18, P. aeruginosa cells B-2099/18, A. baumannii B-2926/18, E. cloacae K-205, S. aureus 7775, C. albicans AV-85. Microorganisms were isolated during investigation of outbreaks of infectious diseases on the instructions of Rospotrebnadzor of Rusian Federation in 2012-2016 and tested as follows.

Antibacterial Activity of the Composition “Hexanidine Sept” Against Test Strains of Pathogens of Nosocomial Infections

During the study, a previously developed methodological approach was used that allows to conduct a comparative analysis of the sensitivity of microorganisms to antibacterial compositions, including antiseptics and disinfectants, in the plankton state and inside biofilms.

It is shown that the composition “Hexanidine Sept” has an antibacterial activity against cells of all used test strains and clinical strains of microorganisms (n=13) in the plankton state (Table 3). It was also found that the composition “Hexanidine Sept” has an ability to inhibit growth of culture of yeast C. albicans inside biofilms.

TABLE 3
Antibacterial activity of the composition “Hexanidine
Sept” against test strains of microorganisms in
planktonic form and inside biofilms

| | | MBC of the composition | |
|---|---|---|---|
| | | “Hexanidine Sept”, % of the | |
| | | concentrate “Hexanidine” | |
| No | Strains of microorganisms | Plankton cells | Biofilms |
| 1 | K. pneumoniae ATCC 700603 | 0.005 | 0.31 |
| 2 | K. pneumoniae B-2523/18 | 0.002 | 0.31 |
| 3 | P. aeruginosa ATCC 27853 | 0.02 | 1.25 |
| 4 | P. aeruginosa B-2099/18 | 0.02 | 2.5 |
| 5 | A. baumannii ATCC 15308 | 0.04 | 0.16 |
| 6 | A. baumannii B-2926/18 | 0.04 | 0.31 |
| 7 | E. cloacae ATCC BAA-2468 | 0.02 | 0.16 |
| 8 | E. cloacae K-205 | 0.02 | 0.31 |
| 9 | S. aureus No 906 | 0.005 | 0.08 |
| 10 | S. aureus 7775 | 0.01 | 0.16 |
| 11 | C. albicans ATCC 10231 | 0.04 | 0.02 |
| 12 | C. albicans AV-85 | 0.08 | 0.02 |
| 13 | M. terrae TCM 1450 | 0.005 | 0.04 |

### Example 9. Evaluation of Resistance Formation to the Composition Based on Solubilized Chlorhexidine Base “Hexanidine Sept” from Nosocomial Pathogens

Formation of resistance to the composition “Hexanidine Sept” was studied on the type strains K. pneumoniae ATCC 700603, P. aeruginosa ATCC 27853, A. baumannii ATCC 15308, E. cloacae ATCC BAA-2468, S. aureus No. 906, C. albicans ATCC 10231 and M. terrae TCM 1450, representatives of the types of microorganisms that are among the most characteristic pathogens of nosocomial infections.

Under conditions of selective pressure of stepwise increasing concentrations of the compositions “Hexanidine Sept” no stable variants of strains were obtained for 20 days (8 passages) and no one strain of the above mentioned microorganisms has developed a level of resistance to the antiseptic exceeding its working concentration, which indicates the possibility of its long-term use.

Thus, a composition was created that have unique ability—universality of action toward all types of microorganisms, including those inside biofilms, as well as an unusually low increase in resistance to them from the main pathogens of hospital-acquired infections, which will ensure their long-term use to solve this urgent problem of modem medicine.

Variants of disinfecting compositions have improved quality:
ability to affect full range of microorganisms;
low cost of production due to the use of chlorhexidine base, which is a semi-product in the production of other kinds of this substance;
ease of production.

1. A method of solubilization of chlorhexidine base, comprising encapsulating chlorhexidine base in a presence of an amine oxide.

2. The method according to claim 1, wherein an amino acid is added during encapsulation to enhance solubilization process.

3. The method according to claim 2, wherein the amine oxide is one from the following group: N-lauryl-N, N-dimethylaminoxide, N,N-dimethyldodecan-1-amine oxide, Lauryldimethylamine oxide, Tallowbis(2-hydroxyethyl)amine oxide, C12-18-alkyldimetylamine oxide, and the amino acid is one from the following group: leucine, tyrosine, serine, glutamine, asparagine, phenylalanine, alanine, lysine, arginine, histidine, glycine, cysteine, valine, proline, methionine, threonine, hydroxylysine.

4. An antiseptic or disinfectant composition comprising chlorhexidine base and an amine oxide, wherein the antiseptic composition inhibits growth of bacterial or fungal cells and is soluble in water or alcohol.

5. The antiseptic or disinfectant composition according to claim 4, wherein the composition additionally comprises an amino acid.

6. The antiseptic or disinfectant composition according to claim 5, wherein the amine oxide is N-lauryl-N, N-dimethylaminoxide; the amino acid is one from the following group: leucine, tyrosine, serine, glutamine, asparagine, phenylalanine, alanine, lysine, arginine, histidine, glycine, cysteine, valine, proline, methionine, threonine, hydroxylysine.

7. The antiseptic or disinfectant composition according to claim 5, wherein the composition is intended for therapeutic or prophylactic use against Micobacterium tuberculosis strains, including at least one of the following strain: poly-resistant MDR strains, extremely resistant XDR strains and pan-resistant PDR strains.

8. The antiseptic or disinfectant composition according to claim 5, wherein the composition disrupts biofilms formed from bacterial or fungal cells.

9. The antiseptic or disinfectant composition according to claim 5, wherein the composition is a concentrate and further comprises glycerin, a dye and water in the following ratio of components, mass fraction %:
Chlorhexidine base-0.1-1.0%,
Amine oxide-0.2-5.0%,
Amino acid-0.05-2.0%,
Glycerin-0.0-5.0%,
Dye-0.01-0.1%,
Water—up to 100%.

10. The antiseptic or disinfectant composition according to claim 9, wherein the amine oxide is N-lauryl-N, N-dimethylaminoxide; the amino acid is one from the following group: leucine, tyrosine, serine, glutamine, asparagine, phenylalanine, alanine, lysine, arginine, histidine, glycine, cysteine, valine, proline, methionine, threonine, hydroxylysine, and the dye is selected from the following group: an anthraquinone dye, carmoisine (E122) or triarylmethane dye (E133).

11. The antiseptic or disinfectant composition according to claim 5, wherein the composition is a concentrate and further comprises polyethylene glycol, a dye and water in the following ratio of components, mass fraction %:
Chlorhexidine base-0.5-5.0%,
Amine oxide-5.0-30.0%,
Polyethylene glycol-0.2-3.0%,
Amino acid-0.05-2.0%,
Dye-0.01-0.1%,
Water—up to 100%.

12. The antiseptic or disinfectant composition according to claim 5, wherein the composition is a concentrate and further comprises an alcohol, a dye and water in the following ratio of components, mass fraction %:
Chlorhexidine base-0.5-65.0%,
Amine oxide-0.2-5.0%,
Amino acid-0.1-2.0%,
Dye-0.01-0.1%,
Alcohol-5-50%.

13. The antiseptic or disinfectant composition according to claim 12, where the amine oxide is N-lauryl-N, N-dimethylaminoxide, the alcohol is ethyl alcohol, and the dye is E133 dye.

14. A method for inhibiting growth of bacterial or fungal cells, comprising contacting said cells with an antiseptic or disinfectant composition comprising chlorhexidine base and an amine oxide for a sufficient time, wherein the antiseptic or disinfectant composition is soluble in water or alcohol.

15. The method according to claim 14, wherein the antiseptic or disinfectant composition additionally comprises an amino acid, a quaternary ammonium compound (QAC), an anti-corrosion additive and an acidity regulator.
